(19) United States
(12) Patent Application Publication
(10) Pub. No.: US 2011/0251563 A1
(43) Pub. Date: Oct. 13, 2011

(54) Totally Implantable Venous Device

(76) Inventors: Po-Jen Ko, Taipei City (TW); Ching-Yang Wu, Linkou Township (TW); Yun-Hen Liu, Guishan Shiang (TW)

(21) Appl. No.: 12/758,087

(22) Filed: Apr. 12, 2010

(51) Int. Cl. A61M 25/01

(52) U.S. Cl. 604/175

(57) ABSTRACT

A totally implantable venous device includes a port comprising an arcuate housing having a recess on an inner surface and an opening being in communication with the recess, the opening having a flat mouth, an internal chamber, and a base formed of hard plastic and being capable of preventing a needle from inserting through; a catheter comprising a fastening member at one end; a sleeve complementarily mounted on a front portion of the fastening member; and a sleeving member complementarily mounted on the remaining portion of the fastening member. The recess and the opening form a through hole having an enlarged intermediate portion. The fastening member has a truncated trapezoidal shape. The sleeve, the sleeving member, and the fastening member form a watertight structure at the recess and the opening. The catheter and the fastening member are formed of the same material.

### BACKGROUND OF THE INVENTION

1. Field of Invention

The invention relates to totally implantable venous devices and more particularly to such a totally implantable venous devices with improved characteristics including easy insertion, easy holding, and durability.

2. Description of Related Art

Conventional totally implantable venous devices are disadvantageous due to the following reasons: It is not easy to handle. In detail, only experienced doctors have higher success rate. However, experienced doctors often complain its difficulties. Catheter fracture is susceptible. The implant seat or catheter is required to be removed from the patient if such occurs. This can cause great pain to the patient. Further, it can reduce the medical effects.

Conventionally, a totally implantable venous device comprises a port, a catheter, and a locking nut. In assembly, the catheter is inserted into the connection portion of the port and then put the locking nut on to fix the catheter. The connection portion of catheter is susceptible of fracture due to structural weaknesses. An integral type of totally implantable venous device is commercially available. However, the insertion is more difficult if the small caliber of the blood vessel or sharp angle between cephalic vein and axillary vein are encountered.

The conventional totally implantable venous devices may be found mechanical failure due to more components. In other words, component simplification means improved performance.

Referring to FIG. 7, a conventional totally implantable venous device 1 is shown in cross-section. A conventional totally implantable venous device consists of 3 components, including a port which contains a connecting portion, a catheter, and a locking nut. Because of strength difference between the catheter and other components, catheter fracture may be expected. In addition, the implant seat 1 of the port has a small outer surface 2 which limits the adjustment angle of the needle 6 and the metal wire 3 contained in the needle 6. Further, both the needle 6 and the metal wire 3 are difficult to insert through the outer surface 2, the chamber 5, and the catheter 4. This may lead to some difficulties when the catheter is adjusted over the guide metal wire 3. Therefore, these disadvantages lead to the further structural improvement.

### SUMMARY OF THE INVENTION

It is therefore one object of the invention to provide a totally implantable venous device comprising a port comprising an arcuate housing having a recess on an inner surface and an opening being in communication with the recess, the opening having a flat mouth, an internal chamber, and a base formed of hard plastic and being capable of preventing a needle from inserting through; a catheter comprising a fastening member at one end; a sleeve complementarily mounted on a front portion of the fastening member; and a sleeving member complementarily mounted on the remaining portion of the fastening member; wherein the recess and the opening form a through hole having an enlarged intermediate portion; wherein the fastening member has a truncated trapezoidal shape; wherein the sleeve, the sleeving member, and the fastening member together form a watertight structure at the recess and the opening; wherein the catheter and the fastening member are formed of the same material; and wherein a metallic guide wire is contained in a hollow need which is operable to insert through the housing to contact the fastening member.

The above and other objects, features and advantages of the invention will become apparent from the following detailed description taken with the accompanying drawings.

### BRIEF DESCRIPTION OF THE DRAWINGS

FIG. 1 is an exploded view of a preferred embodiment of totally implantable venous device according to the invention;

FIG. 2 is a perspective view of the assembled implant seat;

FIG. 3 is a cross-sectional view of the catheter with a metallic guide wire inserted thereinto;

FIG. 4 is an environmental view of the implant seat placed upon the body of a patient;

FIG. 5 schematically depicts a section of the metallic guide wire;

FIG. 6 is a perspective view of an puncture sheath of the invention; and

FIG. 7 is a cross-sectional view of a conventional totally implantable venous device.

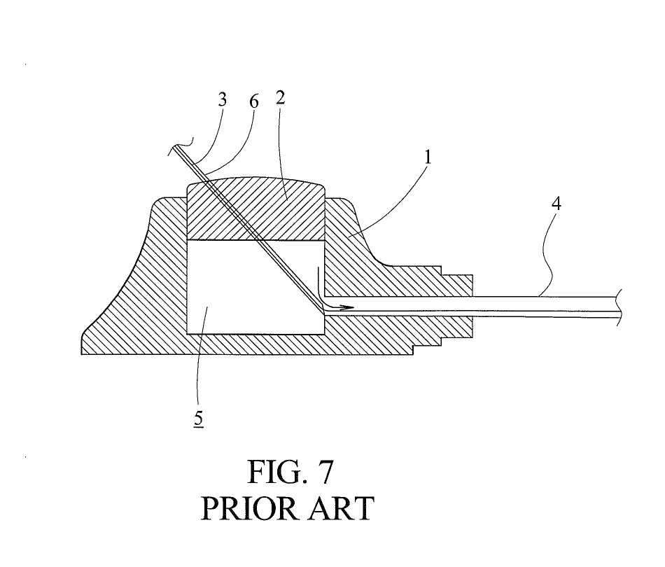

### DETAILED DESCRIPTION OF THE INVENTION

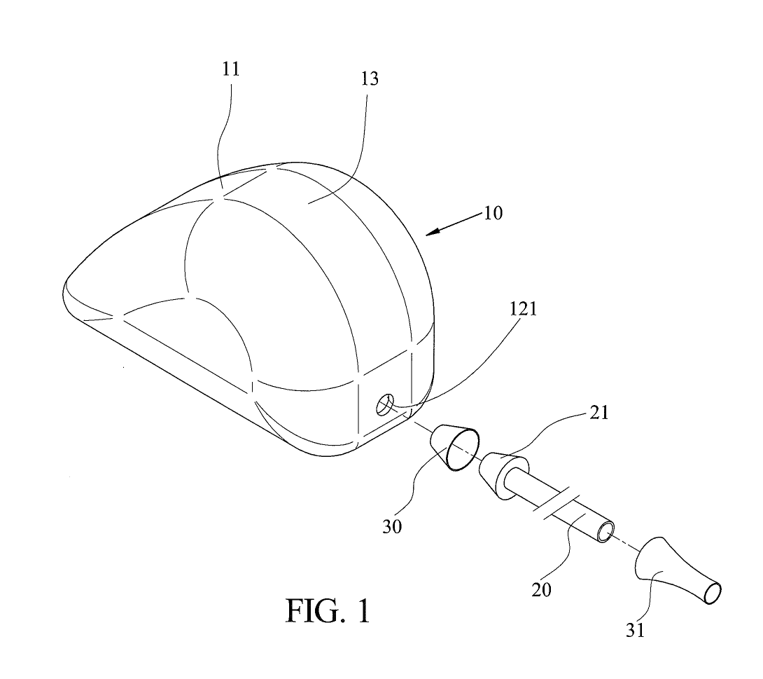

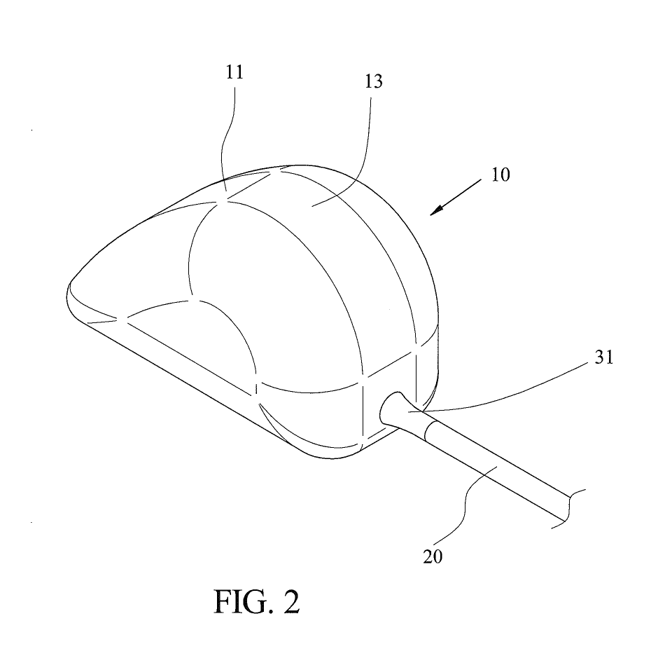

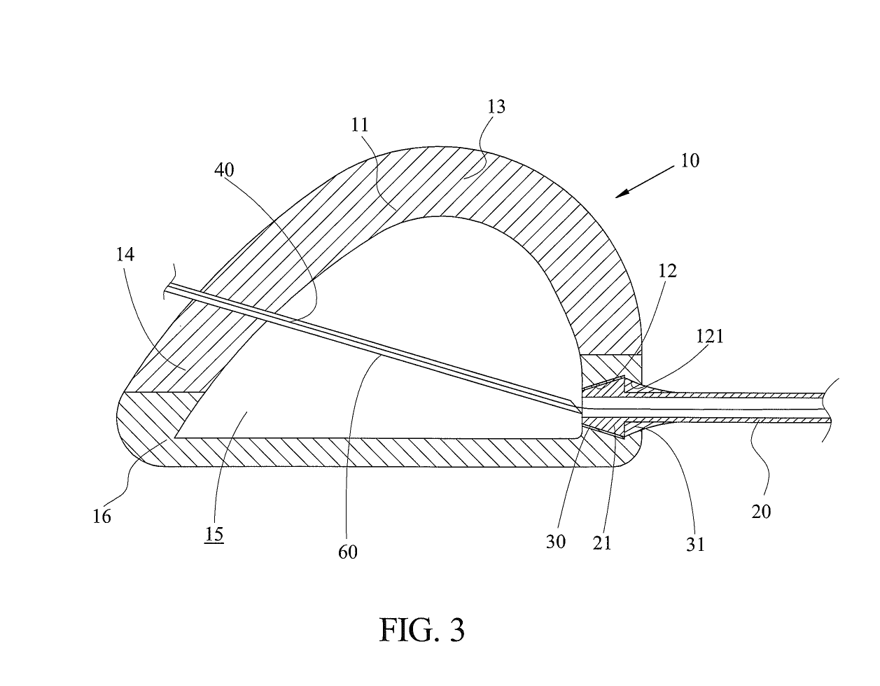

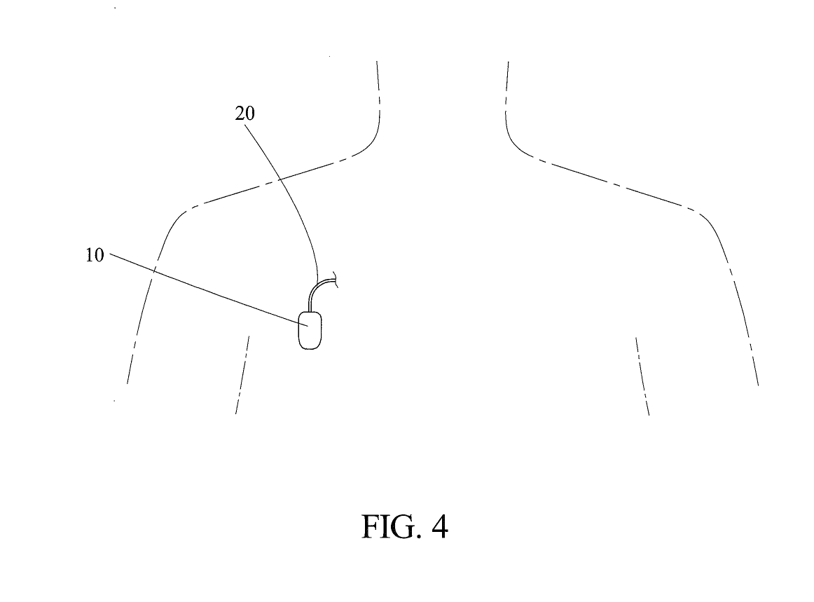

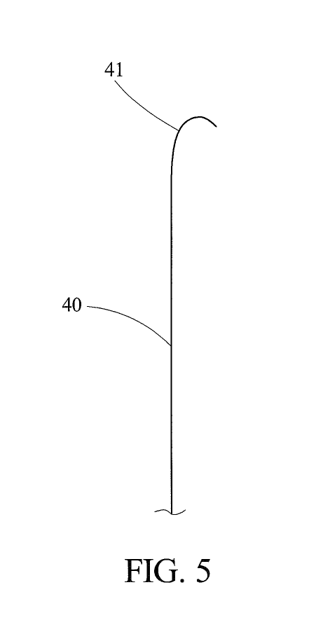

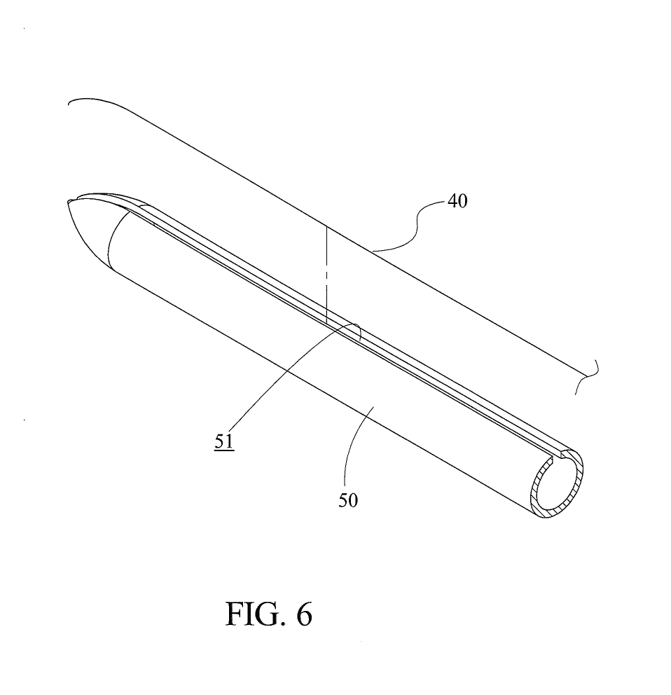

Referring to FIGS. 1 to 6, a totally implantable venous device in accordance with a preferred embodiment of the invention comprises the following components as discussed in detail below.

A hollow port 10 has an ergonomic shape for ease of holding by hand. The port 10 has a housing 11 of arcuate surface 13 for allowing a needle to insert through from various angles, an internal chamber 15, and a base 16 formed of hard plastics. The base 16 is structurally strong to prevent a needle from inserting through.

A catheter 20 comprises an enlarged head 21 of truncated trapezoidal shape at one end. A thin sleeve 30 of also truncated trapezoidal shape is formed of silicone. The sleeve 30 has an independent sleeving member 31 having a flare end. A recess 12 is formed on an inner surface of an upward extending frame of the base 16 and an opening 121 having a narrow flat mouth is formed on an outer surface of an upward extending frame of the base 16. The recess 12 and the opening 121 together form a hole through the upward extending frame of the base 16. The sleeve 30 is complimentarily put on the enlarged head 21 in the recess 12 and the sleeving member 31 is put on the rear portion of the enlarged head 21 in the opening 121. The sleeve 30, the sleeving member 31, and the enlarged head 21 together form a watertight structure at the recess 12 and the opening 121.

Preferably, the sleeving member 31 can withstand a pressure in the range of 20 and 30 Bar. Still preferably, an upper portion 14 of the port 10 is formed of silicone the same as the catheter 20. Advantageously, the flat mouth of the opening 121 can reduce friction of the inserted catheter 20 and facilitate the insertion.

In assembly, the sleeve 30 is put on the enlarged head 21 and the sleeving member 31 is put thereon subsequently. The large intermediate portion of the through hole consisting of the recess 12 and the opening 121 can fasten the catheter 20 after assembly. Further, the catheter 20 and its enlarged head 21 are formed of the same material and together they are not susceptible of breaking. Moreover, the arcuate surface 13 of the port 10 facilitates the holding by hand in use.

As a comparison, conventional totally implantable venous devices have rectangular bodies with four edges being in contact with the skin, i.e., frictional contact. This undesirably may cause erosion of overlying skin. This is particularly true to cancer patients. Further, exposed implant seats are required to be removed or reimplanted to a new site. In this invention, the specific arcuate shape leads to less pressure and less friction between the implant seat and overlying skin.

Both totally implantable venous devices of the prior art and the invention are secured onto the chest. The implant seat of the invention has its port 10 being different from that of the prior art. As described above, the port 10 has an arcuate housing 11 which has the advantages of reducing friction with the skin and easy holding by hand.

The totally implantable venous device of the invention may cooperate with a metallic guide wire 40 which has a layer formed thereon. The metallic guide wire 40 may further cooperate with a hollow needle 60, i.e., passing through the needle 60. The needle 60 may insert into the port 10 to contact the catheter 20 in use.

Preferably, the metallic guide wire 40 has a length of about 50 cm.

The puncture sheath 50 has a longitudinally slit 51. The puncture sheath 50 can be utilized for small vessel cannulation. When small caliber of vessel is encountered, the metallic guide wire 40 is used to cannulate the vessel and the puncture sheath 50 is mounted via longitudinally slit 51 to create subcutaneous tunnel. After subcutaneous tunnel is created, catheter 20 is advanced over-the-wire to the adequate position under intra-operative fluoroscopy. Finally, catheter 20 is connected to the port 10.

The puncture sheath 50 is particularly useful for artificial blood vessels with a small caliber or a large bent angle.

Conventional metallic guide wires are spiral. Portions of the metallic guide wires may be left within the body if there is no surrounding layer. The left metallic guide wire 40 is difficult of removing. Advantageously, the metallic guide wire 40 of the invention have a smooth surrounding layer. Hence, the metallic guide wire 40 of the invention are not susceptible of being left within the body after being pulled from the blood vessel. Further, it is easy to remove dirt from the metallic guide wire 40 of the invention. The conventional metal wire has a J-shaped end. Hence, it may jam within the chamber 15 of the port 1 after insertion, i.e., difficult of being removed. As an improvement, the metallic guide wire 40 of the invention has a slightly bent end. Hence, it is easy to remove same from the chamber 15 of the port 1 after insertion.

The arcuate housing 11 of the port 10 of the invention can increase injection area. Hence, it is easy to adjust angles of the cooperating metallic guide wire 40 and the needle 60. Further, it is easy to insert the metallic guide wire 40 through the chamber 15 of the port 10 to contact the catheter 20. Moreover, the metallic guide wire 40 may be inserted through the catheter 20 out of the port 10.

On the contrary, the conventional implant seat has a small injection area. Hence, adjustment angle of the metal wire is limited. It is thus difficult to insert the metal wire through the chamber of the port and out of the port.

The metallic guide wire 40 of the invention has a slightly bent end 41. In comparison, the typical metal wire has a J-shaped end. Hence, the metallic guide wire of the invention is easy to use.

The portion of connecting the catheter 20 to the port 10 is on an inner surface of the port 10. Hence, it is not easy to bend or even break the catheter 20. On the contrary, the conventional connection point is disposed externally of the port. Hence, it is susceptible of breaking. The catheter 20 and its enlarged head 21 are formed integrally. On the contrary, the conventional catheter and its head are separate members. The upper portion 14 of the port 10 is formed of silicone the same as the catheter 20. Moreover, the connecting portion of the port 10 and the catheter 20 is thin so as to allow vibration of the catheter 20.

The port 10 of the invention is ergonomic in shape. Hence, it is easy to hold the port 10. Further, its arcuate surface facilitates needle insertion. Furthermore, its insertion angle can be adjusted easily. In addition, the arcuate port 10 of the invention has less friction with the skin.

While the invention herein disclosed has been described by means of specific embodiments, numerous modifications and variations could be made thereto by those skilled in the art without departing from the scope and spirit of the invention set forth in the claims.

What is claimed is:

1. A totally implantable venous device comprising:
a port comprising an arcuate housing having a recess on an inner surface and an opening being in communication with a recess, the opening having a flat mouth, an internal chamber, and a base formed of hard plastic and being capable of preventing a needle from inserting through;
a catheter comprising a fastening member at one end;
a sleeve complementarily mounted on a front portion of the fastening member; and
a sleeving member complementarily mounted on the remaining portion of the fastening member;
wherein the recess and the opening form a through hole having an enlarged intermediate portion;
wherein the fastening member has a truncated trapezoidal shape;
wherein the sleeve, the sleeving member, and the fastening member together form a watertight structure at the recess and the opening;
wherein the catheter and the fastening member are formed of the same material; and
wherein a metallic guide wire is contained in a hollow need which is operable to insert through the housing to contact the fastening member.

2. The totally implantable venous device of claim 1, further comprising an elongated puncture sheath having a longitudinally slit for guiding an insertion of the metallic guide wire.
